(19) United States
(12) Patent Application Publication
(10) Pub. No.: US 2011/0082334 A1
(43) Pub. Date: Apr. 7, 2011

(54) ENDOSCOPIC INSTRUMENT

(75) Inventors: Martin Dolt, Knittlingen (DE); Hubert Völlinger, Rastatt (DE)

(73) Assignee: RICHARD WOLF GMBH, Knittlingen (DE)

(21) Appl. No.: 12/891,326

(22) Filed: Sep. 27, 2010

(30) Foreign Application Priority Data
Sep. 29, 2009 (DE) 10 2009 043 652.9

(51) Int. Cl. A61B 1/00

(52) U.S. Cl. 600/104

(57) ABSTRACT

The endoscopic instrument has at least one component that heats up during the operation of the instrument and is equipped with means for automatically registering the use or non-use state of the instrument. The instrument has a control, which actuates the at least one component in a power-reduced fashion or switches off the at least one component during the non-use of the instrument, and which actuates the component with the power provided for the use or switches on the component during the use of the instrument.

### BACKGROUND OF THE INVENTION

The invention relates to an endoscopic instrument having at least one component that heats up during the operation of the instrument.

Endoscopic instruments are becoming ever more powerful and smaller, but the heat generation within the instrument also increases as the power density rises, be it directly due to the electrical/electronic components that heat up because of electricity flowing through them, as for example, the CCD sensor for recording the image in a video endoscope, or else due to optical components as a result of absorption, coupling or reflection losses of optical radiation. Dissipating this lost heat is complicated; measures that often cannot be used or can only be used with insufficient effect due to the limited space are required for dissipating heat. This also holds true for fans and blowers, which are typically only used in the stationary part of such an endoscopic system and moreover generate noise, which is undesirable. However, increasingly there are problems as the temperature rises in the endoscope. In addition to the increased temperature being detrimental to the electronic image acquisition and processing as a result of a significant increase in noise, during prolonged use temperatures can be generated on the external sides, which cause discomfort or pain in the case of, for example, hand-held components.

In order to avoid this, the prior art already discloses a provision for switching off or reducing the power in endoscopic systems in order to reduce the additional heat load. This typically is brought about via switches on the control unit, i.e., it always requires manual intervention by the therapist, distracting him from the actual goal of diagnosis or treatment.

Moreover, the prior art discloses automatically conducting such a reduction in power or automated switching off when the instrument is put into the holder provided therefor, the holder being equipped with a micro-switch or a magnetic switch for carrying out this function. This also assumes that the instrument is set aside by the treating person as intended. However, what often happens in practice is that the instrument is only set aside on the patient, on the table or the like during an intervention, so that it can be quickly picked up again, or because an ergonomic workplace design does not allow the provision of an appropriate holding device.

### BRIEF SUMMARY OF THE INVENTION

Against this backdrop, it is the object of the invention to develop an endoscopic instrument of the aforementioned type, such that heat development within the instrument can be reduced, where possible without having a negative impact on its capability.

The endoscopic instrument according to the invention, which contains one or more components that heat up during the operation of the instrument, is provided with means for automatically registering the use or non-use of the instrument and has a control, which actuates the at least one component that heats up during the operation of the instrument in a power-reduced fashion or switches off the at least one component when the non-use of the instrument is registered, and which actuates the component that heats up during the operation of the instrument with the power provided for the use or switches on the component the use of the instrument is registered.

Hence, it is a basic idea of the present invention to determine, by automatically registering the state of the instrument, when the instrument is actually in use or when it is in non-use, i.e. only set aside or on the way to being used. For this, according to the invention, means for automatically registering the use or non-use of the instrument are provided. Here it typically suffices to register one of the two use states, i.e. to register whether the instrument is in use or in non-use. The actuating or switching function for controlling the at least one component that heats up during the operation of the instrument can suitably be effected both when the time of non-use and the time of use is registered. In practice, the assumption can be made that whenever the instrument is not in use, it is in a state of non-use, and vice versa; the control should be designed appropriately.

Depending on instrument and application, there are various means available for registering use or non-use. In a multiplicity of instruments, it should be possible to register the use or non-use by a position sensor within the instrument. In this context, reference is made in a purely exemplary fashion to instruments for a laryngoscopic application, i.e., more particularly to laryngoscopes for diagnostic purposes, which are typically applied when the patient is seated. The shaft of the instrument is typically aligned substantially horizontally during use. If a sufficiently large angle-tolerance range is fixed, merely registering the position of the instrument can determine whether or not the instrument is being used as intended. Then, in the case of non-use, when the instrument has just been set aside or is not inserted, the control can be used to switch off or at least reduce the power of one or more components of the instrument that heat up during the operation of the instrument, so that the heat loss is reduced and hence the instrument is heated less in times of non-use.

Depending on the application, a two-dimensional (2D) position sensor already suffices for this, but the use of a three-dimensional (3D) position sensor is particularly advantageous, which three-dimensional position sensor can also be constructed from two two-dimensional (2D) position sensors arranged at 90° to one another. The three-dimensional (3D) position sensor naturally allows registering the use or non-use even in those instruments that can be used in positional orientations with greater variance.

Alternatively, or if need be additionally, an accelerometer arranged within the instrument can also be used to register the use or non-use of the instrument. By using a suitable electronic evaluation, this accelerometer can be used to determine whether the instrument is situated in the hand of a therapist, or on a table or a body part of the patient. The accelerometer can be used to register the typical movements of the therapist in the case of manual handling and distinguish these from the instrument resting on an object or from the movements when the instrument is situated on a body part of the patient, and hence use or non-use can be determined.

An electronic evaluation arrangement is typically provided for evaluating the sensor signal, the evaluation arrangement being signal-connected to the sensor, or the sensors if a plurality thereof are provided. It is particularly advantageous for this evaluation arrangement to form part of the control, which then also actuates the power or switches on or off the components that heat up during the operation of the instrument, according to the use or non-use being registered.

Such an evaluation arrangement advantageously has an input device that can be used to enter data, which data define the use or non-use situations and fix tolerances in respect thereof. When using a position sensor as described at the outset, positional data can typically be entered into the evaluation arrangement that defines the use or non-use position. The inputs can be, for example, specified as angular deviations from predefined spatial axes and can expediently be adjusted individually by the user, at least within bounds.

In particular, if at least one accelerometer is used, at least one filter, or if need be also a plurality of filters are provided in the evaluation arrangement according to one advantageous embodiment of the invention, in order to differentiate between a use and a non-use movement.

Modern endoscopic instruments, which have electronic image acquisition and can be connected to an external illumination source via optical fibers, typically have a stationary control unit in addition to the actual endoscopic instrument, which control unit is electrically connected to the endoscopic instrument and feeds the latter. According to a further embodiment of the invention, the control, which advantageously also comprises the evaluation arrangement and undertakes the power actuation of the component or components in the instrument that heat up during the operation of the instrument, is part of this control unit used to feed the instrument at least with energy, typically in the form of electrical energy and/or optical energy in the form of illumination light.

According to an advantageous embodiment of the invention, in which the component that heats up during the operation of the instrument arranged within the endoscopic instrument is an optical component in the path of the illumination light, the control does not influence the optical system but influences the control of the electrical power of the illumination source, with the latter being actuated with less power or even switched off in the case of non-use.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a much-simplified schematic illustration of a laryngoscope in a use position, with a position sensor arranged therein according to an embodiment of the invention;

FIG. 2 is a similar illustration of the laryngoscope according to FIG. 1 in a non-use position; and

FIG. 3 is a similar illustration of the laryngoscope according to FIG. 1 in a further non-use position.

### DETAILED DESCRIPTION OF THE INVENTION

The laryngoscope illustrated by way of the figures is one in which the image is acquired by an image sensor (not illustrated) arranged in a hand grip portion 2, is subsequently processed electronically and transmitted via a transmission line 3 to a stationary control unit 4 and from there to a monitor. An illumination source is also arranged in the control unit 4, which guides illumination light via waveguides situated in the transmission line 3 into the shaft 5 of the laryngoscope 1, which illumination light emerges there from the distal end and illuminates, e.g., the vocal chords to be observed.

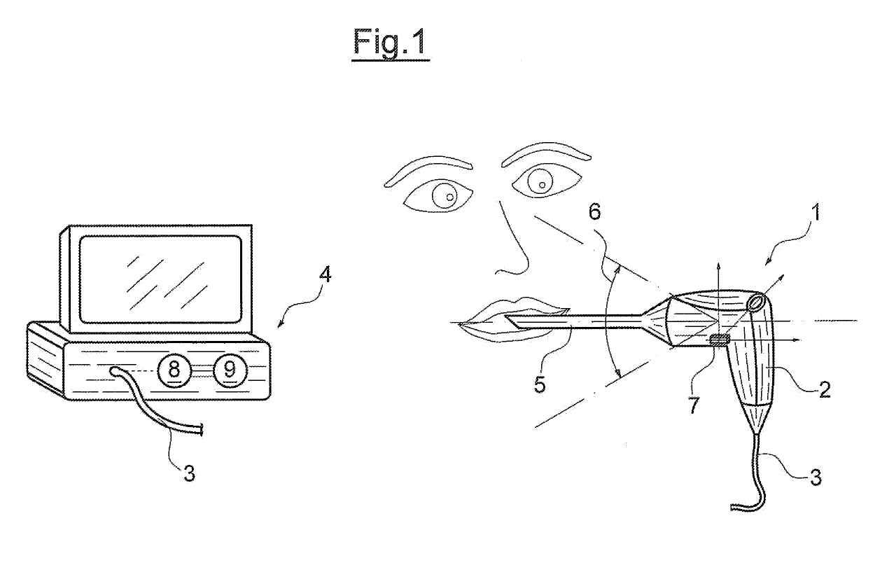

Even though the illustrated laryngoscope 1 can be used for different examination purposes, it basically is always used in the position illustrated schematically in FIG. 1 with a horizontally aligned shaft 5, which position results during the insertion of the instrument if the patient sitting in front of the therapist has an open mouth. In the process, certain angular deviations from the illustrated horizontal position are possible, for example inclines of 30° obliquely upward or obliquely downward. The use position of the instrument 1 is defined within this spatial position, which is specified in an exemplary fashion in FIG. 1 by the angle 6. It can be assumed that there is non-use in all positions deviating therefrom, i.e., an intended use is not possible and thus components that heat up during the operation of the instrument may also be switched off or partly switched off.

In order to register this, a 3D gravitational sensor 7 is provided in the hand grip portion 2 of the laryngoscope, the output signal of which sensor is fed via the transmission line 3 to the control unit 4 and there to an evaluation unit 8 which determines whether the laryngoscope 1 with its shaft 5 is in the angular range specified by 6. If this is the case, the downstream control 9 receives a signal that characterizes the use state, as a result of which the control 9 releases all illumination, recording and other functions of the laryngoscope 1 in the control unit 4.

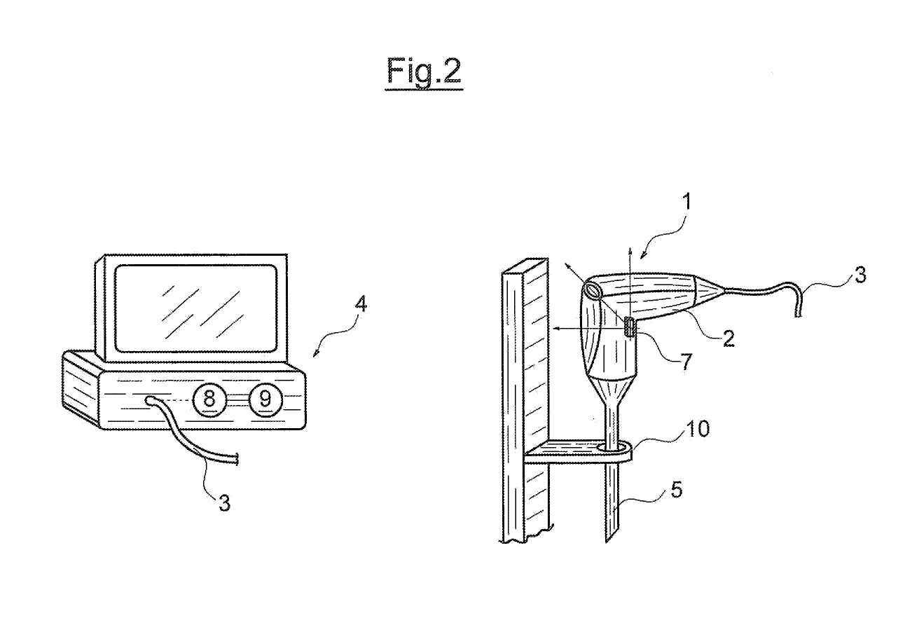

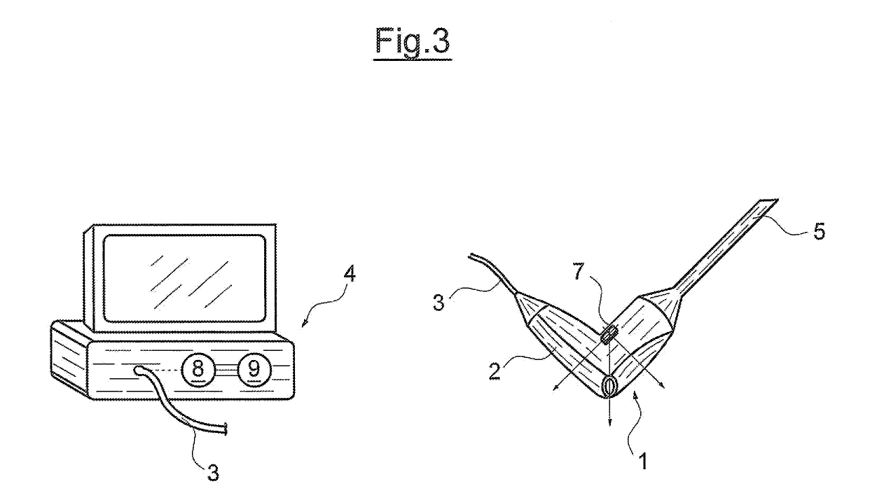

By contrast, if the laryngoscope 1 is stored in a holder 10 in the case of non-use, as illustrated in FIG. 2, the shaft 5 points vertically downward and the hand grip portion 2 adjoins the former in the upward direction. In this position, the laryngoscope is clearly outside of the angular range 6 illustrated in FIG. 1, which angular range permits pivot movements around the center of gravity or any other fixed point of the instrument by plus/minus 30° from the horizontal in each case. The positional information is transmitted by means of the sensor 7 to the evaluation unit 8, which identifies this positional state as a non-use state and emits an appropriate signal to the control 9, which thereupon switches off the illumination within the control unit 4 or reduces it to a minimum, and which moreover switches off the image sensor situated within the laryngoscope 1. This is correspondingly brought about in the position illustrated in FIG. 3, in which the laryngoscope has been set aside on a planar surface and the shaft and hand grip portion 2 are situated in approximately the same horizontal plane. This position is transmitted by the sensor 7 to the evaluation unit 8, which identifies this position as a non-use state and, as described with the aid of FIG. 2, actuates the control 9 with a signal corresponding to the non-use state.

Only once the user transfers the laryngoscope 1 into a position illustrated in FIG. 1, in which the hand grip portion 2 is arranged such that the transmission line 3 basically goes off downward and the shaft 5 has a substantially horizontal position with a tolerance angular range 6, does the evaluation unit 8 signal the use-state as a result of the sensor signal, whereby the control 9 activates the illumination situated in the control unit 4 and switches on the image recording unit.

It is understood that this applies to the present exemplary embodiment and the specific application of a laryngoscope, and that the positional orientation should be modified in accordance with the usage purpose in other endoscopic instruments in order to be able to determine in the evaluation unit 8, with the aid of the positional orientation, whether the instrument 1 is in a use or non-use position.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1.-9. (canceled)

10. An endoscopic instrument (1) comprising at least one component that heats up during operation of the instrument, means (7, 8) for automatically registering a use or non-use of the instrument (1), and a control (9) which actuates the at least one component in a power-reduced fashion or switches off the at least one component during the non-use of the instrument (1) and which actuates the at least one component with power provided for the use or switches on the at least one component during the use of the instrument (1).

11. The endoscopic instrument according to claim 10, wherein the means for automatically registering the use or non-use comprises a position sensor (7) within the instrument (1).

12. The endoscopic instrument according to claim 11, wherein the position sensor is a 3D gravitational sensor.

13. The endoscopic instrument according to claim 11, wherein the sensor (7) is signal-connected to an evaluation arrangement (8), which is signal-connected to the control (9).

14. The endoscopic instrument according to claim 11, wherein the sensor (7) is signal-connected to an evaluation arrangement (8), which forms part of the control (9).

15. The endoscopic instrument according to claim 10, wherein the means for automatically registering the use or non-use of the instrument includes an evaluation arrangement (8) into which positional data defining the use or non-use position can be entered.

16. The endoscopic instrument according to claim 10, wherein the means for automatically registering the use or non-use comprises an accelerometer within the instrument.

17. The endoscopic instrument according to claim 16, wherein the means for automatically registering the use or non-use of the instrument includes an evaluation arrangement (8), and wherein the evaluation arrangement includes filters for differentiating between a use and a non-use movement.

18. The endoscopic instrument according to claim 10, wherein the control (9) is part of a stationary control unit (4) that feeds the instrument (1) at least with energy.

19. The endoscopic instrument according to claim 10, wherein the at least one component is an optical component in an illumination light path, and wherein electrical power of an illumination source for the optical component is controlled in a power-reduced or switched off position of the instrument.
